(19) United States
(12) Patent Application Publication
(10) Pub. No.: US 2015/0087988 A1
(43) Pub. Date: Mar. 26, 2015

(54) ULTRASOUND TRANSDUCER ARRAYS

(71) Applicant: GENERAL ELECTRIC COMPANY, Schenectady, NY (US)

(72) Inventors: Warren Lee, Niskayuna, NY (US); Bruno Hans Haider, Ballston Lake, NY (US); Stephen Dodge Edwardsen, Niskayuna, NY (US); Geir Ultveit Haugen, Oslo (NO); Scott Cogan, Clifton Park, NY (US); Chester Saj, Niskayuna, NY (US); Christopher Yetter, Niskayuna, NY (US); Bjornar Sten-Nilsen, Oslo (NO); Shinichi Amemiya, Hachiouji-shi (JP); Charles Edward Baumgartner, Niskayuna, NY (US)

(73) Assignee: GENERAL ELECTRIC COMPANY, Schenectady, NY (US)

(21) Appl. No.: 14/032,392

(22) Filed: Sep. 20, 2013

(51) Int. Cl. A61B 8/00

(52) U.S. Cl. CPC A61B 8/4494
USPC 600/459

(57) ABSTRACT

An ultrasound transducer array for an ultrasound probe is presented. The ultrasound transducer array includes a support structure. Further, the ultrasound transducer array includes a plurality of electro-acoustic modules coupled to the support structure, wherein each of the plurality of electro-acoustic modules comprises at least one matrix acoustic array and an interconnect element, wherein each of the plurality of electro-acoustic modules is interchangeable on the support structure so as to adapt to one or more shapes of the ultrasound probe, and wherein each of the plurality of electro-acoustic modules operates in a manner substantially identical to each other of the plurality of electro-acoustic modules.

### BACKGROUND

Embodiments of the present disclosure relate generally to ultrasound transducers, and more particularly to a system and method for assembling an ultrasound transducer array using electro-acoustic modules.

Ultrasound transducers are used extensively for ultrasound imaging of an object. Particularly, in a medical field, the ultrasound transducers are typically used to obtain a high quality image of a region within a patient. Further, this high quality image may be used for diagnosing the patient.

An ultrasound transducer typically includes transducer arrays that are generally used for transmission and reception of ultrasonic or acoustic waves. These acoustic waves are further processed to obtain the image of the object. In general, the transducer arrays may be flat transducer arrays or convex transducer arrays. The flat transducer arrays are commonly used in cardiac imaging while, the convex transducer arrays are used in other diagnostic applications, such as abdominal imaging.

In a conventional ultrasound transducer, the flat transducer arrays are formed by fabricating large arrays on a single substrate. However, this type of fabricating process includes additional steps, such as lamination and dicing of large parts, which further results in more scrap materials. This in turn increases the cost of the ultrasound transducers.

In addition, the convex transducer arrays are formed by fabricating a large array in a flat configuration and subsequently bending the large array into its final form. Typically, the large array is in direct contact with beam forming electronics or an application specific integrated circuit (ASIC). Thus, while bending the large array, the beam forming electronics or ASIC are also bent along with the large array. Further, bending the beam forming electronics or ASIC may induce sufficient internal stresses, which in turn alters the ASIC functionality and/or reliability. Therefore, it is preferred to fabricate or form the convex transducer array without bending the electronics or ASICs.

Thus, there is need for an improved method and system for fabricating/assembling ultrasound transducer arrays.

### BRIEF DESCRIPTION

In accordance with one embodiment described herein, an ultrasound transducer array for an ultrasound probe is presented. The ultrasound transducer array includes a support structure. Further, the ultrasound transducer array includes a plurality of electro-acoustic modules coupled to the support structure, wherein each of the plurality of electro-acoustic modules comprises at least one matrix acoustic array and an interconnect element, wherein each of the plurality of electro-acoustic modules is interchangeable on the support structure so as to adapt to one or more shapes of the ultrasound probe, and wherein each of the plurality of electro-acoustic modules operates in a manner substantially identical to each other of the plurality of electro-acoustic modules.

In accordance with a further aspect of the present disclosure, an electro-acoustic module for an ultrasound transducer array is presented. The electro-acoustic module includes a base unit including an acoustic backing and a heat sink, wherein the heat sink is configured to detachably couple to a support structure of the ultrasound transducer array. Further, the electro-acoustic module includes an ASIC layer individually coupled to the base unit. Also, the electro-acoustic module includes a flex interconnect disposed on the ASIC layer and electrically coupled to a circuit board. In addition, the electro-acoustic module includes a matrix acoustic array disposed on the flex interconnect and comprising a plurality of stack elements at least partially separated by a vertical gap, wherein at least one narrow stack element is positioned between two wide stack elements, wherein the at least one narrow stack element has a width extending horizontally between the vertical gaps that is lesser than a width of the wide stack elements.

### DRAWINGS

These and other features, aspects, and advantages of the present invention will become better understood when the following detailed description is read with reference to the accompanying drawings in which like characters represent like parts throughout the drawings, wherein:

FIG. 1 is a perspective view of a transducer array having electro-acoustic modules, in accordance with aspects of the present disclosure;

FIG. 2 is a perspective view of an electro-acoustic module, in accordance with aspects of the present disclosure;

FIG. 3 is a bottom view of the electro-acoustic module, in accordance with aspects of the present disclosure;

FIG. 4 is a side view of the transducer array depicting an enlarged portion of an acoustic array, in accordance with aspects of the present disclosure;

FIG. 5 is a side view of the electro-acoustic module, in accordance with one embodiment of the present disclosure;

FIG. 6 is a side view of electro-acoustic modules assembled on a non-planar support structure, in accordance with one embodiment of the present disclosure;

FIG. 7 is a cross section of the electro-acoustic module, in accordance with aspects of the present disclosure;

FIG. 8 is a side view of a transducer array mounted on a convex support structure, in accordance with aspects of the present disclosure;

FIG. 9 is a diagrammatical representation of the transducer array, in accordance with one embodiment of the present disclosure;

FIG. 10 is a diagrammatical representation of an electro-acoustic module, in accordance with one embodiment of the present disclosure;

FIG. 11 is a diagrammatical representation of the transducer array, in accordance with another embodiment of the present disclosure

FIG. 12 is a diagrammatical representation of an electro-acoustic module, in accordance with another embodiment of the present disclosure;

FIG. 13 is a perspective view of an ultrasound probe, in accordance with aspects of the present disclosure;

FIG. 14 illustrates an electro-acoustic module having one design of an elongated flex interconnect, in accordance with aspects of the present disclosure;

FIG. 15 illustrates an electro-acoustic module having another design of an elongated flex interconnect, in accordance with aspects of the present disclosure;

FIG. 16 illustrates a side view of an electro-acoustic module illustrating elements of an acoustic array, in accordance with one embodiment of the present disclosure;

FIG. 17 illustrates a side view of an electro-acoustic module illustrating elements of an acoustic array, in accordance with another embodiment of the present disclosure; and

FIG. 18 illustrates a side view of an electro-acoustic module illustrating elements of an acoustic array, in accordance with yet another embodiment of the present disclosure.

### DETAILED DESCRIPTION

As will be described in detail hereinafter, various embodiments of ultrasound transducer arrays and methods for fabricating the same are presented. The transducer arrays may comprise electro-acoustic modules that are interchangeable and adaptable to a shape of an ultrasound probe. Also, the formation of these transducer arrays yields minimal scrap material, thus reducing the manufacturing cost of the ultrasound probe. Moreover, the transducer arrays can be assembled on a convex structure without bending the electronics or ASIC, which in turn improves the functionality and/or reliability of the ASIC.

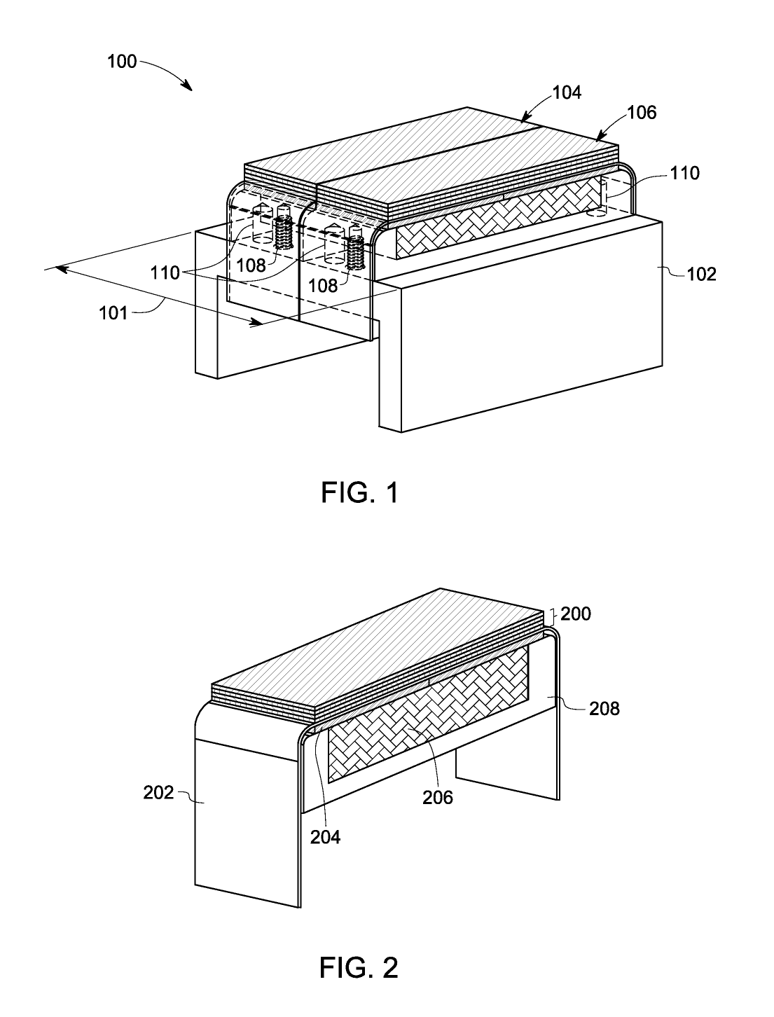

Turning now to the drawings and referring to FIG. 1, a transducer array having electro-acoustic modules, in accordance with aspects of the present disclosure, is depicted. The transducer array 100 is typically used to transmit ultrasonic or acoustic waves towards an object (not shown in FIG. 1). In response to transmitting the ultrasonic waves, the transducer array 100 may receive reflected or attenuated ultrasonic waves from the object. Further, these received ultrasonic waves are processed to obtain an ultrasonic image of the object. In one embodiment, the object may be a region of interest in a patient.

In a presently contemplated configuration, the transducer array 100 includes a support structure 102 and one or more electro-acoustic modules 104, 106 that are coupled to the support structure 102. Each of these electro-acoustic modules 104, 106 may be interchangeable on the support structure 102, and thus, the electro-acoustic modules 104, 106 may not be required to be located in a particular position on the support structure 102. Also, each of the electro-acoustic modules 104, 106 may be similar in size, which aids in easy extensibility, replaceablity, and/or flexibility during design and manufacture of an ultrasound probe. Moreover, the electro-acoustic modules 104, 106 may be tiled or aligned on a planar or non-planar portion of a support structure so as to adapt to a shape of an ultrasound probe. In addition, if one of these electro-acoustic modules 104, 106 is affected or damaged then it may be replaced by a new electro-acoustic module.

In the embodiment of FIG. 1, the transducer array 100 includes two electro-acoustic modules 104, 106 that are detachably coupled to a portion 101 of the support structure 102. The portion 101 of the support structure 102 may be a planar structure that acts as a base or spine for the electro-acoustic modules 104, 106. In one embodiment, the support structure 102 may be a non-planar structure, such as a convex structure. Further, the electro-acoustic modules 104, 106 may be smaller in size compared to the support structure 102, which in turn aids in designing and manufacturing a desired ultrasound probe. For example, the electro-acoustic modules 104, 106 may be arranged on the support structure 102 to form a portable ultrasound probe.

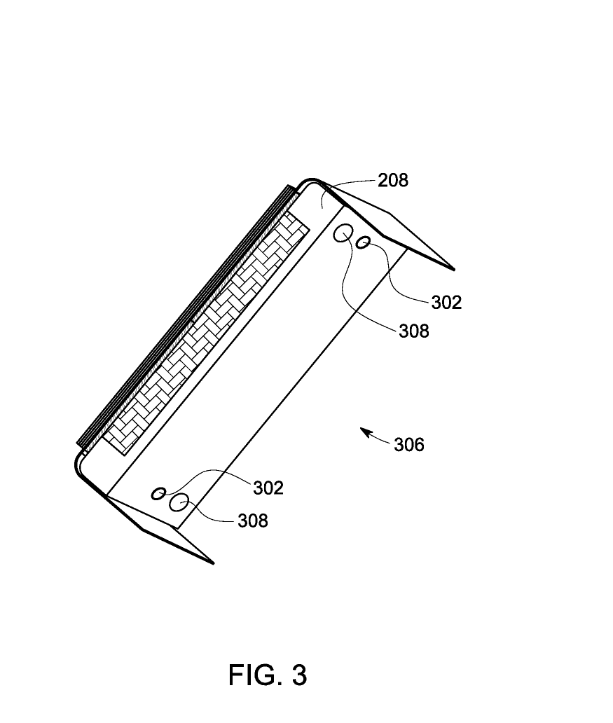

In addition, the electro-acoustic modules 104, 106 may be arranged on one or more types of the support structure to conform to the shape of the ultrasound probe. For example, if the electro-acoustic modules 104, 106 are arranged on a flat portion 101 of the support structure 102, a flat transducer array may be formed. In another example, if the electro-acoustic modules 104, 106 are arranged on a convex portion of the support structure, a convex transducer array may be formed (see FIG. 8). In addition, the support structure 102 includes protruding members 108 and pins 110 that are used for coupling the electro-acoustic modules 104, 106 to the support structure 102. The aspect of coupling the electro-acoustic modules 104, 106 to the support structure 102 is explained in greater detail with reference to FIG. 3. It should be noted that the transducer array 100 may include any number of electro-acoustic modules, and is not limited to the number of electro-acoustic modules shown in FIG. 1.

Furthermore, as depicted in FIG. 2, each of the electro-acoustic modules 104, 106 includes a matrix acoustic array 200, a flex interconnect 202, one or more application-specific integrated circuits (ASIC) 204, an acoustic backing 206, and a heat sink 208. The matrix acoustic array 200 is configured to send one or more acoustic waves towards the object. In response, the matrix acoustic array 200 may receive the reflected acoustic waves from the object. These acoustic waves may have a frequency in a range from about 0.5 MHz to about 25 MHz. In one embodiment, the matrix acoustic array 200 includes single or multiple rows of electrically and acoustically isolated transducer elements. Each of these transducer elements may be a layered structure including at least a piezoelectric layer and an acoustic matching layer. In one embodiment, the matrix acoustic array 200 may include micromachined ultrasound transducers, such as capacitive micromachined ultrasonic transducers (cMUTs) and/or piezoelectric micromachined ultrasonic transducers (pMUTs).

As will be appreciated, an electrical pulse is applied to electrodes of the piezoelectric layer, causing a mechanical change in the dimension of the piezoelectric layer. This in turn generates an acoustic wave that is transmitted towards the object. Further, when the acoustic waves are reflected back from the object, a voltage difference is generated across the electrodes that are then detected as a received signal. Thereafter, the received signal from each of the transducer elements in the acoustic array 200 is combined and processed by the ASIC 204.

Moreover, the matrix acoustic array 200 is coupled to the flex interconnect 202 that is used for providing electrical connection between the acoustic array and signal processing electronics or circuit board (not shown in FIG. 2) that is disposed within a body of the ultrasound probe. In one example, the flex interconnect 202 may be used to communicate the electrical pulses between the piezoelectric layer and the signal processing electronics.

Further, the ASIC 204 is coupled to the acoustic backing 206 and the heat sink 208, as depicted in FIG. 2. The acoustic backing 206 may be configured to absorb and/or scatter the acoustic waves or energy that is transmitted in a direction away from the object being scanned. Particularly, the acoustic waves are generated by the piezoelectric layer. Further, a portion of the generated acoustic waves may be reflected from structures or interfaces behind the transducer array. These acoustic waves may combine with the acoustic waves that are reflected from the object, which in turn reduces the quality of the ultrasonic image of the object.

To avoid the above problem, the acoustic backing 206 may be positioned beneath the ASIC 204 to attenuate or absorb the acoustic waves that are propagated in the reverse direction to the object. In one example, the acoustic backing 206 may include acoustic backing materials that are combinations of a high-density acoustic scatterer, such as tungsten metal, and/or a soft acoustic absorbing material, such as silicone, in a matrix of an epoxy or a polyurethane. In another example, the backing material may comprise an epoxy filled graphite foam which has the added advantage of having a high thermal conductivity to draw heat away from the ASIC. Also, the heat sink 208 may be configured to absorb or dissipate the heat generated in the electro-acoustic module. In one embodiment, the heat sink 208 along with the acoustic backing 206 may be configured to absorb the heat generated in the electro-acoustic module.

In one embodiment, the heat sink 208 includes one or more apertures 302 on a bottom surface 306 of the heat sink 208, as depicted in FIG. 3. The one or more apertures 302 are configured to receive protruding members 108 from the support structure 102 and in one embodiment the apertures 302 may be threaded. In one example, the protruding members 108 may include screws that are inserted into apertures 302 that are threaded for coupling the electro-acoustic module 104 to the support structure 102. Also, the heat sink 208 includes one or more alignment apertures 308 on the bottom surface 306 for receiving one or more pins 110 from the support structure 102. Particularly, while coupling the electro-acoustic module 104 to the support structure 102, tool pins 110 on a top side of the support structure 102 are inserted into corresponding alignment apertures 308 on the bottom surface 306 of the heat sink 208. Thereafter, the electro-acoustic module 104 may be adjusted to avoid any misalignment of the transducer array 100. After adjusting or aligning the position of the electro-acoustic module 104 on the support structure 102, the protruding members 108, such as screws are inserted into the apertures 302 of the heat sink 208 to fasten the electro-acoustic module 104 to the support structure 102. In another embodiment, the electro-acoustic module may include one or more protruding members and the support structure may include one or more apertures. Further, each of the protruding members may be inserted into a corresponding aperture to fasten and/or align the electro-acoustic module to the support structure. In yet another embodiment, the electro-acoustic module may be coupled to the support structure by using a glue film/layer between the electro-acoustic module and the support structure. Also, in one more embodiment, the support structure may have a receiving pocket shape that matches with a shape of the heat sink in the electro-acoustic module. Further, when the electro-acoustic module is placed on the support structure, the heat sink may be secured to the support structure so as to fasten the electro-acoustic module to the support structure. Additionally, in one other embodiment, the electro-acoustic modules may be magnetically coupled to the support structure. It may be noted that the electro-acoustic modules may be coupled to the support structure by using any similar fastening/coupling mechanism, and is not limited to the mechanism described above.

Thus, by using the electro-acoustic modules 104, 106, the transducer array 100 may be assembled and conformed to the shape of the ultrasound probe. Also, the electro-acoustic modules 104, 106 may be easily adjusted on the support structure 102 to avoid any misalignment of the transducer array 100.

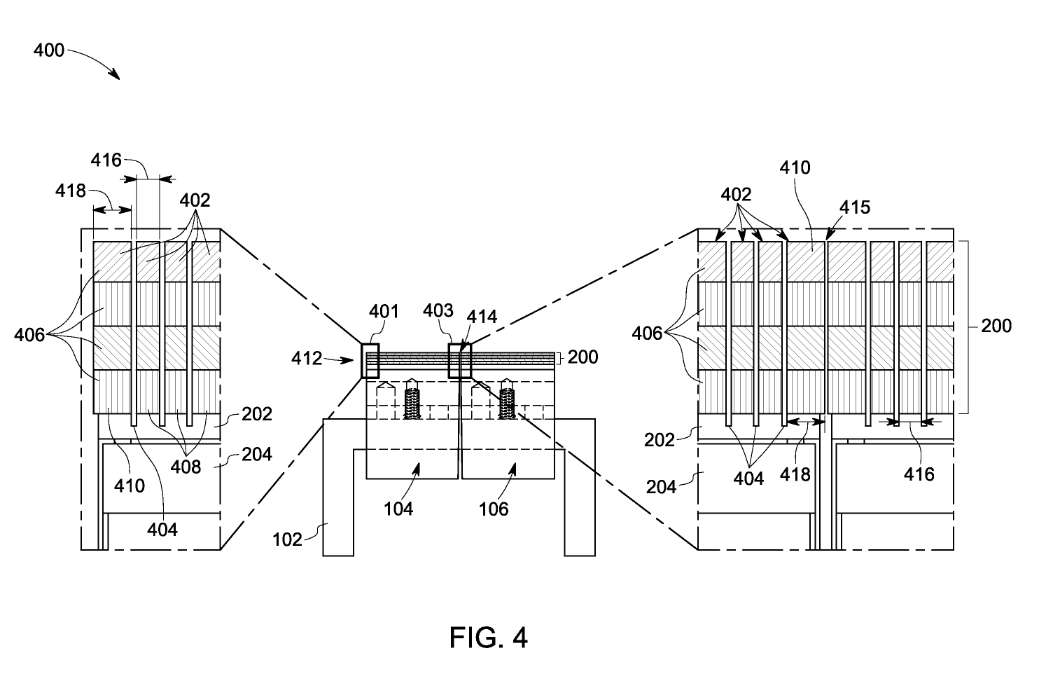

Referring to FIG. 4, a side view of a transducer array 400 illustrating elements of an acoustic array, in accordance with aspects of the present disclosure, is depicted. For ease of understanding, the transducer array 400 is described with reference to the components of transducer array 100 of FIGS. 1-3. The transducer array 400 includes two electro-acoustic modules 104, 106 that are disposed adjacent to each other on a support structure 102. Portions 401, 403 of these two electro-acoustic modules 104, 106 are enlarged to illustrate a structure of a matrix acoustic array 200. Particularly, the enlarged portions 401, 403 depict the matrix acoustic array 200, a flex interconnect 202, and an ASIC 204.

Further, the matrix acoustic array 200 includes acoustic elements 402 that are separated by a vertical gap 404, as depicted in FIG. 4. Each of the acoustic elements 402 is formed by a stack of layers 406 that are used for sending acoustic waves towards an object and receiving the reflected acoustic waves from the object. Furthermore, the acoustic elements 402 include narrow stack elements 408 and wide stack elements 410. Also, the width 416 of the narrow stack elements 408, which is extended horizontally between the vertical gap 404, is lesser than the width 418 of the wide stack elements 410.

Moreover, the wide stack elements 410 are positioned at sides of the acoustic array 104, while the narrow stack elements 408 are positioned between the two wide stack elements 410. For example, a first wide stack element 410 is disposed at a left edge 412 of the acoustic array 104 and the second wide stack element 410 may be disposed at a right edge 414 of the acoustic array 104. Further, the narrow stack elements 408 are placed between the first and second wide stack elements 410, as depicted in FIG. 4. In one embodiment, the wide stack elements 410 are placed at the edges 412, 414 to reduce a vertical gap 415 between adjacent electro-acoustic modules 104, 106 when the electro-acoustic modules 104, 106 are disposed on the support structure 102. By reducing the width of the vertical gap 415, the electro-acoustic modules 104, 106 may receive the reflected acoustic waves with minimal or no signal loss. This in turn improves the quality of the ultrasonic image of the object.

In another embodiment, the two wide stack elements 410 are placed at the edges 412, 414 of the matrix acoustic array 104 such that the two wide stack elements 410 overhang the ASIC 204 in the corresponding electro-acoustic module 104. Particularly, while preparing an individual electro-acoustic module 104, the edges 412, 414 of the electro-acoustic module 104 would otherwise need to be trimmed by using a dicing saw without touching or otherwise affecting the ASIC 204. If the electro-acoustic module 104 includes the wide stack elements 410 at the edges 412, 414, an extra margin may be provided for trimming the electro-acoustic module 104, which in turn aids in dicing the electro-acoustic module 104 without affecting the ASIC 204.

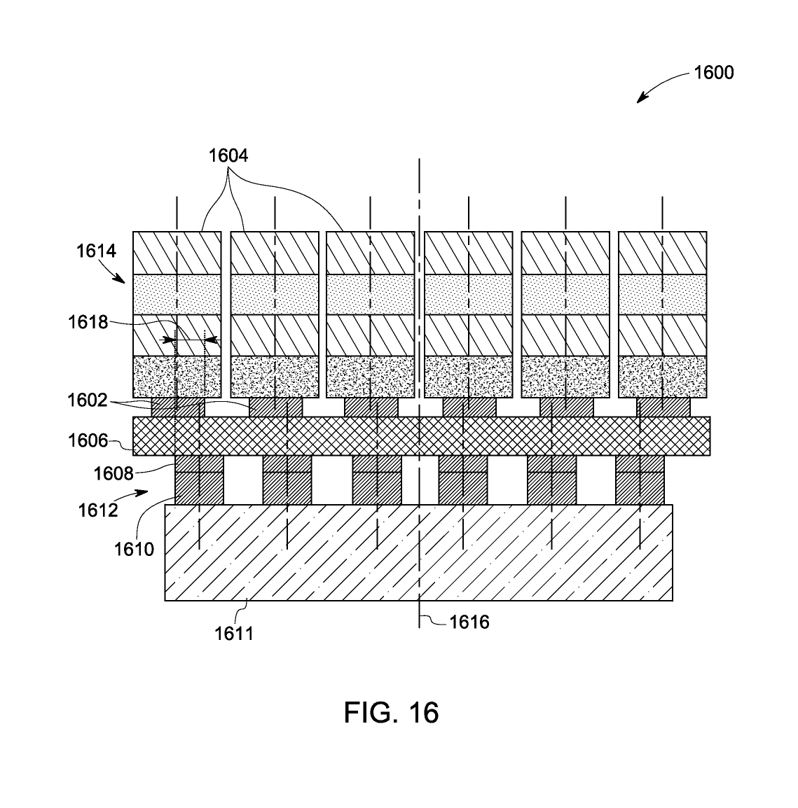

In one embodiment, as depicted in FIG. 16, an electro-acoustic module 1600 includes first pads 1602 that are placed between acoustic elements 1604 and a flex interconnect element 1606. Similarly, the electro-acoustic module 1600 includes second pads 1608 that are placed between an ASIC bump 1610 and the flex interconnect element 1606. It may be noted that the first pads 1602 and the second pads 1608 are referred to as flex circuit pads. Further, the pads 1602, 1608 and the ASIC bump 1610 are used for providing electrical connection between the acoustic elements 1604 and an ASIC 1611.

Also, as depicted in FIG. 16, a pitch of the second pads 1608 is matched with a pitch of the ASIC bump 1610. However, a pitch of the first pads 1602 on a transducer side 1614 is designed to be slightly larger than the pitch of the second pads 1608 on an ASIC side 1612. In one example, if the pitch of the second pads 1608 is ‘x’ then the pitch of the first pads 1602 may be ‘x+y’. It may be noted that though the pads 1602, 1608 have different pitches, the pads 1602, 1608 may still have overlapping region 1618 to provide electrical connection between them. Further, while dicing the acoustic elements 1604, the center saw cut is aligned with the center 1616 of the electro-acoustic module 1600 and the pitch of the saw or dicing cut is matched with the pitch of the first pads 1602 on the transducer side 1614. In one example, the pitch of the dicing cut may be ‘x+y’ which is same as the pitch of the first pads 1602 on the transducer side 1614.

Furthermore, since the pitch of the dicing cut is larger than the pitch of the second pads on the ASIC side, an extra pitch ‘y’ may be accumulated for each dicing cut from the center 1616 to the edge of the electro-acoustic module 1600. Thus, if the electro-acoustic module 1600 has ‘n’ dicing cuts between the center 1616 and the edge of the electro-acoustic module 1600, the acoustic element at the edge of the electro-acoustic module 1600 may be offset from the ASIC bump 1610 by an amount ‘n*y’. This in turn provides extra room to trim the acoustic array without affecting the ASIC or ASIC bump 1610. Also, it may be noted that the acoustic elements 1604 typically will be of uniform size across the electro-acoustic module 1600 irrespective of the pitch of the dicing cut.

Additionally, it may be noted that if the dicing cut is not aligned with the center 1616 of the electro-acoustic module 1600, the acoustic elements 1604 may still have a uniform size. However, an acoustic element 1604 at one edge of the electro-acoustic module 1600 may have an uneven amount of overhang as compared to the acoustic element 1604 at the other edge of the electro-acoustic module 1600.

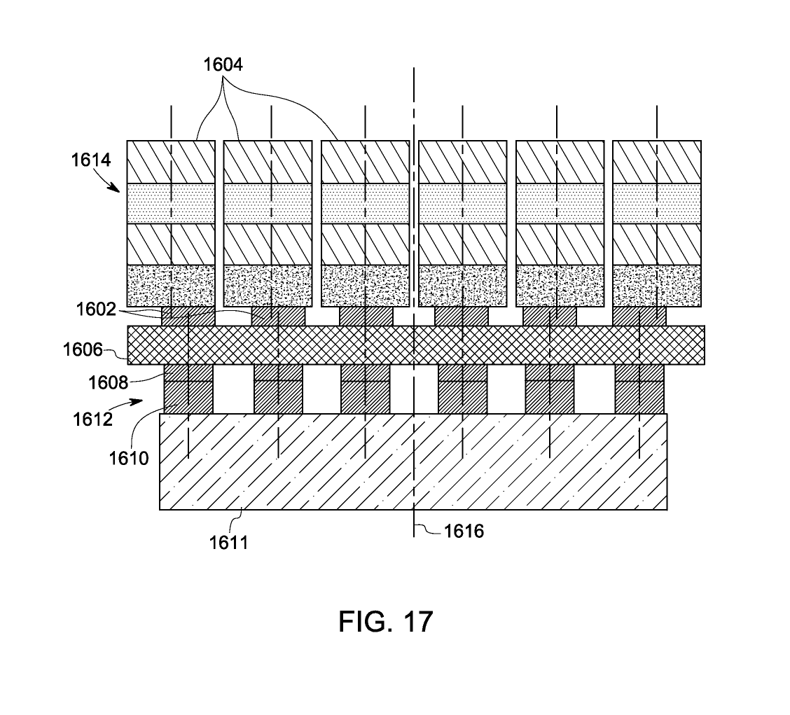

In another embodiment, as depicted in FIG. 17, the pitch of the first pads 1602 and the second pads 1608 may be designed to be the same as the pitch of the ASIC bump 1610. In one example, the pitch of the first pads 1602, the second pads 1608, and the ASIC bump 1610 are designed to be ‘x’. However, the pitch of the dicing cut may be designed to be larger than the pitch of the pads 1602, 1608 and the ASIC bump 1610. For example, if the pitch of the pads 1602, 1608 and the ASIC bump is ‘x’, then the pitch of the dicing cut is designed to be ‘x+y’. Further, while dicing the acoustic elements 1604, an extra pitch ‘y’ may be accumulated for each dicing cut from the center 1616 to the edge of the electro-acoustic module 1600. Thus, if the electro-acoustic module 1600 has ‘n’ dicing cuts between the center 1616 and the edge of the electro-acoustic module 1600, the acoustic element at the edge of the electro-acoustic module 1600 may be offset from the ASIC bump 1610 by an amount ‘n*y’. This again provides extra room to trim the acoustic array without affecting the ASIC or ASIC bump 1610. Further it may be noted that, in this embodiment, the acoustic elements 1604 may not be perfectly aligned with the first pads 1602 on the transducer side 1614. However, the acoustic elements 1604 and the first pads 1602 may have an overlapping region to provide sufficient electrical connection between them. Here again, the acoustic elements 1604 will be of uniform size irrespective of the pitch of the dicing cut.

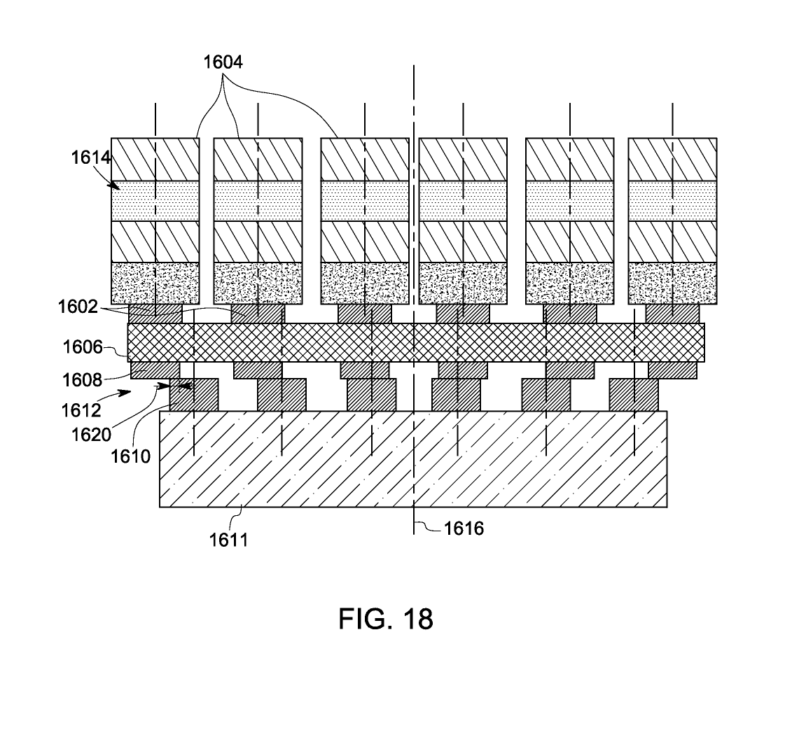

In yet another embodiment, as depicted in FIG. 18, the pitch of the first pads 1602 and the second pads 1608 may be designed to be the same as the pitch of the acoustic elements 1604. In one example, the pitch of the first pads 1602 and the second pads 1608 may be designed to be ‘x+y’. However, the pitch of the ASIC bump 1610 may be designed to be lesser than the pitch of the pads 1602, 1608. For example, if the pitch of the pads 1602, 1608 is ‘x+y’, then the pitch of the ASIC bump 1610 is designed to be ‘x’. Further, while dicing the acoustic elements 1604, an extra pitch ‘y’ may be accumulated for each dicing cut from the center 1616 to the edge of the electro-acoustic module 1600. Thus, if the electro-acoustic module 1600 has ‘n’ dicing cuts between the center 1616 and the edge of the electro-acoustic module 1600, the acoustic element at the edge of the electro-acoustic module 1600 may be offset from the ASIC bump 1610 by an amount ‘n*y’. This in turn provides extra room to trim the acoustic array without affecting the ASIC or ASIC bump 1610. Further, it may be noted that, in this embodiment, the second pads 1608 may not be perfectly aligned with the ASIC bump 1610 on the ASIC side 1612. However, the second pads 1608 and the ASIC bump 1610 may have an overlapping region 1620 to provide sufficient electrical connection between them.

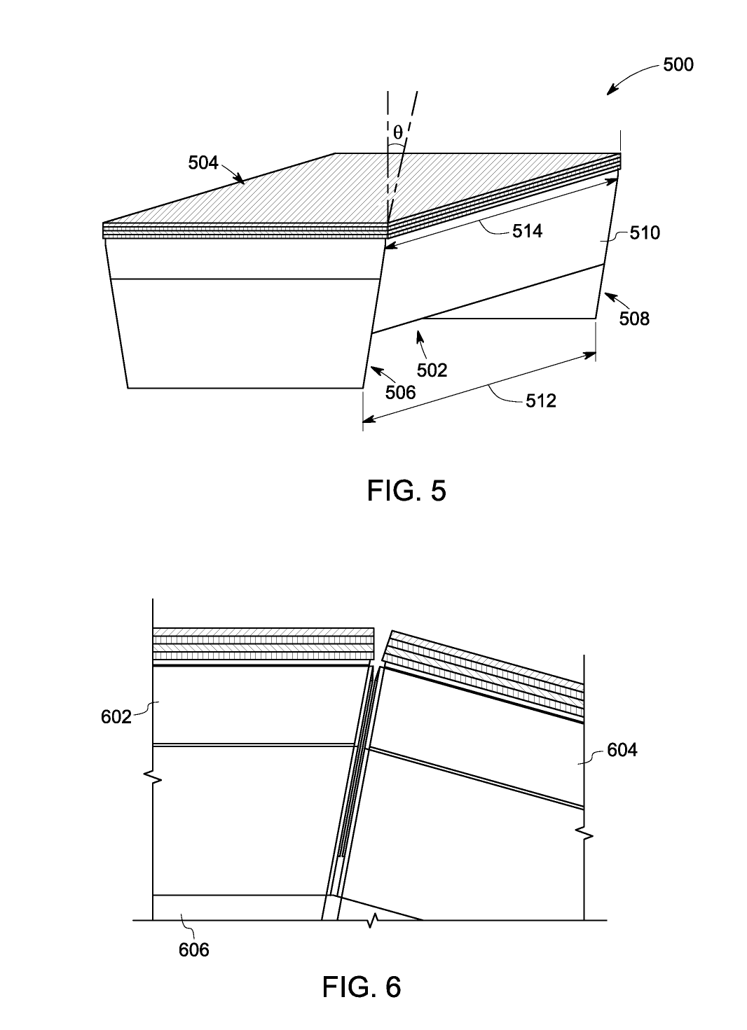

Referring to FIG. 5, a perspective view of the electro-acoustic module, in accordance with an embodiment of the present disclosure, is depicted. For ease of understanding, the electro-acoustic module 500 is described with reference to the components of FIGS. 1-4. If the electro-acoustic modules having flat sides, as depicted in FIG. 1, are positioned on a convex surface, the bottom surfaces of these electro-acoustic modules may interfere with each other, while the top corners of these electro-acoustic modules may be separated by a large gap between the electro-acoustic modules. This large gap may in turn cause loss in the signal received from the object and thereby, an improper image of the object may be obtained.

To overcome the above problem, the sides of the electro-acoustic module 500 are beveled, as depicted in FIG. 5. Particularly, the electro-acoustic module 500 includes a bottom surface 502 and a top surface 504. The bottom surface 502 is adjacent to a support structure 102 when the electro-acoustic module is disposed on the support structure 102. Further, the top surface 504 is opposite to the bottom surface 502 and positioned away from the support structure 102. Also, acoustic energy is emitted from the top surface 504 in a direction that is away from the bottom surface 502. In addition, the electro-acoustic module 500 includes at least two beveled sides 506, 508, as depicted in FIG. 5. In one embodiment, the sides 506, 508 may be beveled at an angle ‘θ’ that is in a range from about 5 degrees to about 20 degrees. Each of the beveled sides 506, 508 may form a surface 510 extending between the bottom surface 502 and the top surface 504. Also, this surface 510 may have a first width 512 at the bottom surface 506 and a second width 514 at the top surface 504 of the electro-acoustic module 500. The first width 512 may be lesser than the second width 514. In one example, the beveled sides 506, 508 may be obtained by tapering the surface 510 from the top surface 504 to the bottom surface 502, as depicted in FIG. 5.

Furthermore, as depicted in FIG. 6, electro-acoustic modules 602, 604 that are similar to the electro-acoustic module 500 are positioned on the convex support structure. Particularly, when the electro-acoustic modules 602, 604 having beveled sides are positioned on the convex support structure, the gap between the electro-acoustic modules 602, 604 may be substantially reduced. Thus, the electro-acoustic modules 602, 604 having beveled sides are used for assembling or forming a convex transducer array which may be further used for diagnostic applications, such as abdominal imaging.

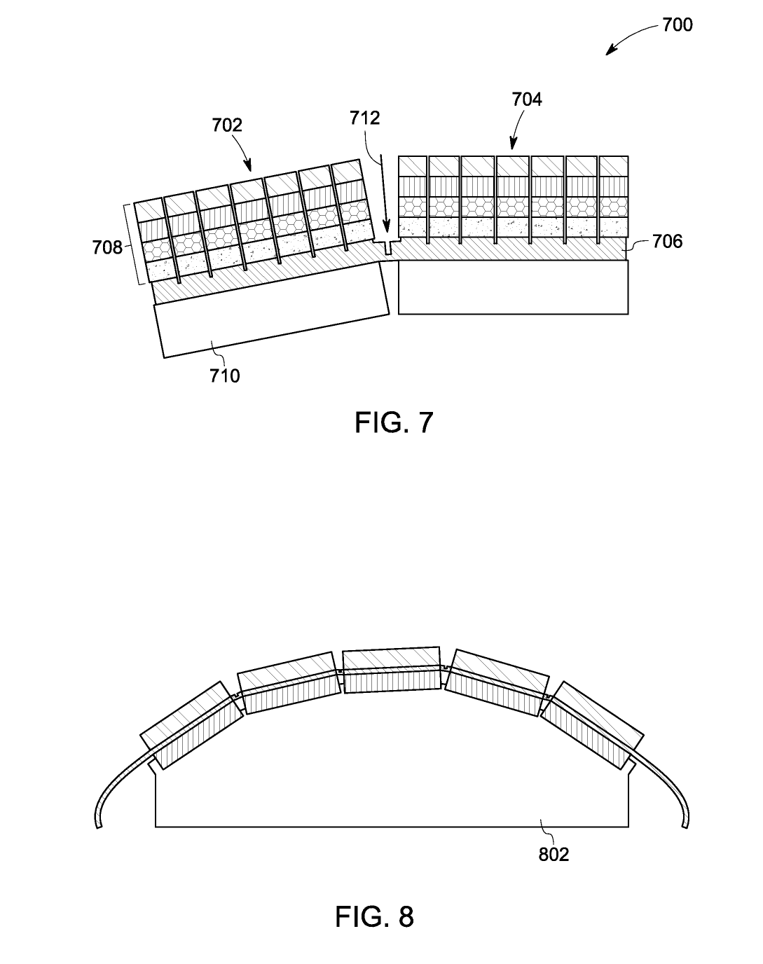

Referring to FIG. 7, a cross section of a transducer array, in accordance with one embodiment of the present disclosure, is depicted. The transducer array 700 includes a plurality of acoustic modules 702, 704 that are interconnected by a flex interconnect 706. Particularly, the acoustic modules 702, 704 are serially coupled to each other via the flex interconnect 706, as depicted in FIG. 7. In one embodiment, a vertical gap 712 may be maintained between the acoustic modules 702, 704 to allow movement of the acoustic modules 702, 704. Further, each of the acoustic modules 702, 704 includes an acoustic array 708, the flex interconnect 706, and an ASIC 710. The acoustic array 708 may be similar to the matrix acoustic array 200 of FIG. 2. Similarly, the ASIC 710 may be similar to the ASIC 204 of FIG. 2.

Moreover, the flex interconnect 706 may be diced in orthogonal azimuth and elevation directions at a region between the acoustic modules 702, 704 to promote bending of the flex interconnect 706. Particularly, the flex interconnect 706 may be partially diced, as depicted in FIG. 7, so that the flex interconnect 706 may be bent to move the acoustic modules 702, 704 in the azimuth direction. In one example, the flex interconnect 706 may be bent to position the acoustic modules 702, 704 on a convex support structure 802, as depicted in FIG. 8. Since the acoustic modules 702, 704 are connected to each other by the flex interconnect 706, the acoustic modules 702, 704 may instead be positioned on a flat structure to form a flat transducer array and the flat transducer array be bent along with the acoustic modules 702, 704 to form a convex transducer array. Thus, single arrangement of acoustic modules 702, 704 may be used to form the flat transducer array or the convex transducer array.

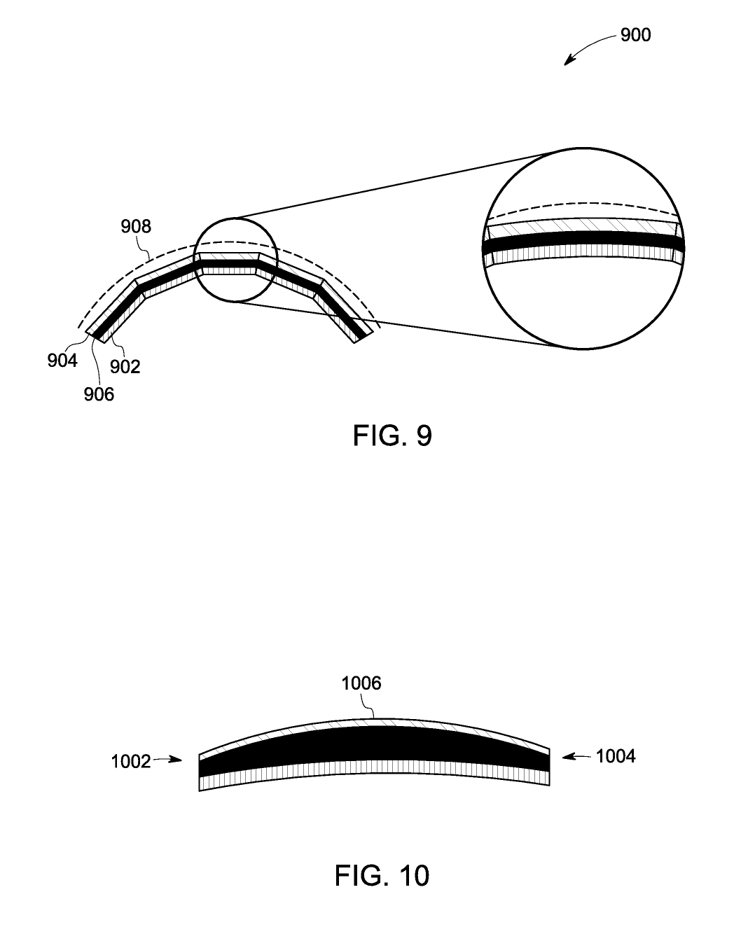

Referring to FIG. 9, a cross section of a transducer array, in accordance with another embodiment of the present disclosure, is depicted. The transducer array 900 is similar to the transducer array 700 of FIG. 7 except that a thickness of a flexible interconnect 906 is varied to conform to a shape of an ultrasound probe. Particularly, the flexible interconnect 906 having variable thickness is coupled between an acoustic array 904 and an ASIC 902 so as to obtain a desired shape of the transducer array 900, as depicted in FIG. 9. In one embodiment, as depicted in FIG. 10, the flex interconnect 902 may have lesser thickness at the edges 1002, 1004 compared to the thickness of the flex interconnect 902 at the center of an acoustic module 1006. This in turn aids in bringing the acoustic array and the ASIC proximate to each other at the edges 1002, 1004 of the acoustic module 1006. Further, by sequentially aligning or positioning such acoustic module 1006, the transducer array 900 that more closely approximates a reference curve 908 may be obtained.

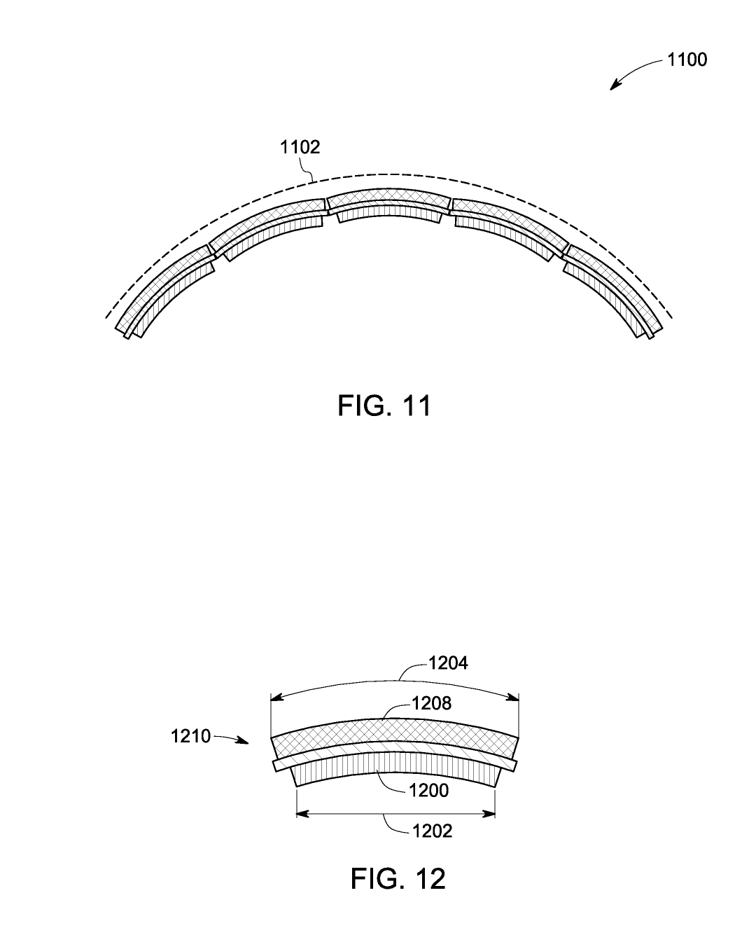

Referring to FIG. 11, a cross section of a transducer array, in accordance with yet another embodiment of the present disclosure, is depicted. The transducer array 1100 is similar to the transducer array 700 of FIG. 7 except that a length of the ASIC is shortened compared to a length of the acoustic array in each acoustic module. Particularly, as depicted in FIG. 12, the length 1202 of the ASIC 1200 is shortened compared to the length 1204 of the acoustic array 1208 in each acoustic module 1210. Further, by positioning such acoustic modules sequentially, a convex transducer array 1100 that more closely approximates a reference curve 1102 may be obtained, as depicted in FIG. 11. In one embodiment, the ASIC in each acoustic module 1200 may be allowed to curve slightly, but at a greater or equal radius of curvature (ROC) than the ultrasound probe is curved to reduce stress on the ASIC. In another embodiment, as depicted in FIG. 11, the ASIC in each acoustic module 1200 may have lesser ROC than the ultrasound probe to obtain a predefined/desired shape of the ultrasound probe.

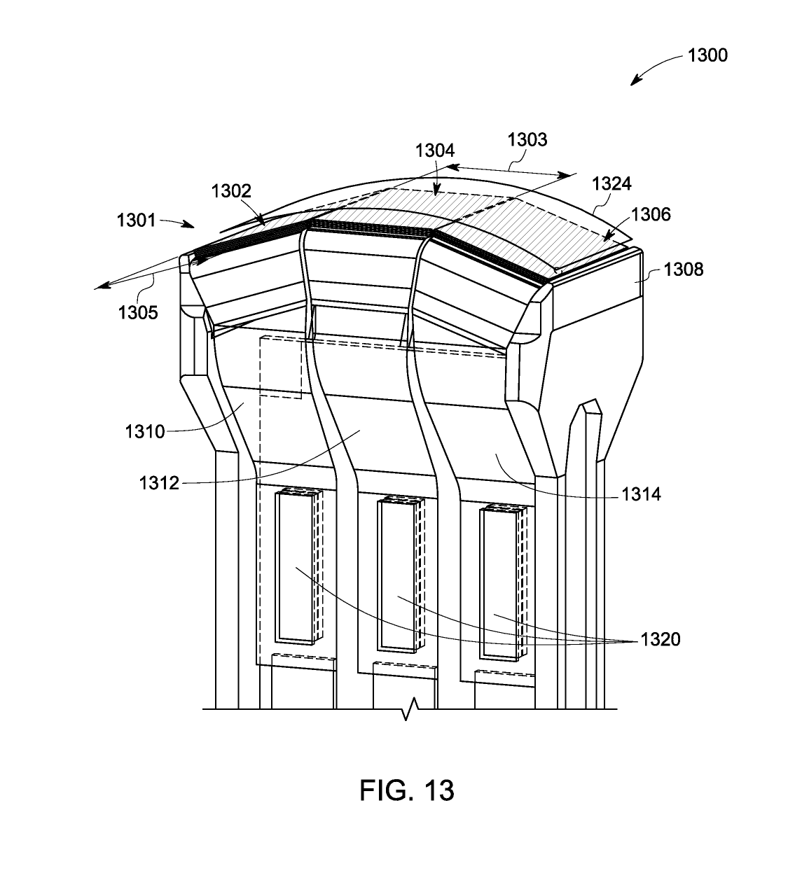

Referring to FIG. 13, a perspective view of an ultrasound probe, in accordance with aspects of the present disclosure, is depicted. The ultrasound transducer probe 1300 includes a transducer array 1301 having three electro-acoustic modules 1302, 1304, 1306 that are coupled to a support structure 1308. Each of the electro-acoustic modules 1302, 1304, 1306 is similar to the electro-acoustic module 500 except that the electro-acoustic modules 1302, 1304, 1306 include elongated flex interconnects 1310, 1312, 1314, as depicted in FIG. 13. Each of the elongated flex interconnects 1310, 1312, 1314 may be flexible and adaptable to provide electrical connection between an acoustic array and a circuit/interface board 1320.

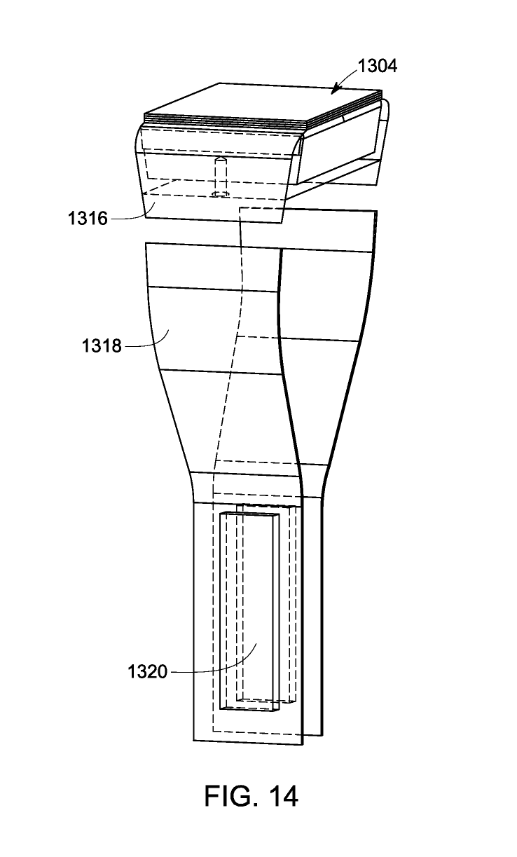

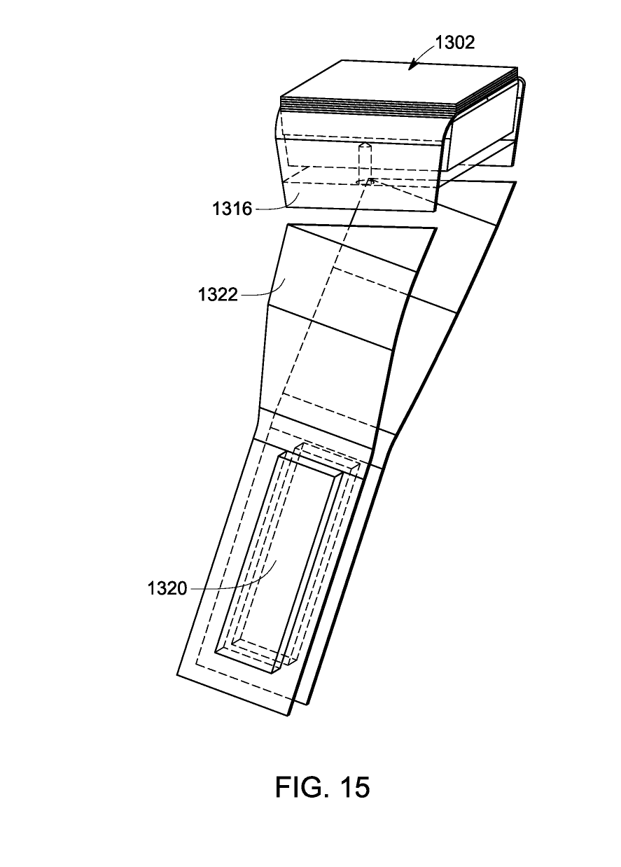

In one embodiment, each of the elongated flex interconnects 1310, 1312, 1314 may be a single strip/element that is connected between the acoustic array and the circuit/interface board 1320. In another embodiment, each of the elongated flex interconnects 1310, 1312, 1314 may include a primary flex interconnect 1316 and a secondary flex interconnect 1318, 1322, as depicted in FIGS. 14 and 15. Particularly, the primary flex interconnect 1316 is disposed between an acoustic array and an ASIC layer, while the secondary flex interconnect 1318, 1322 is electrically coupled between the primary flex interconnect 1316 and a circuit/interface board 1320.

In addition, the secondary flex interconnect 1318, 1322 may have one or more shapes depending on the position of the electro-acoustic modules 1310, 1312, 1314 on a support structure. In one example, if the electro-acoustic module 1304 is on a flat portion 1303 of the transducer array 1301, the secondary flex interconnect 1318 having a straight or unbent shape is coupled to the primary flex interconnect 1316, as depicted in FIG. 14. In another example, the electro-acoustic module 1302 is positioned on a left curved portion 1305 of the transducer array 1301, and thus, a secondary flex interconnect 1322 having a slanted or angled edge is coupled to the primary flex interconnect 1316, as depicted in FIG. 15. Thus, one of different shapes of the secondary flex interconnect 1318, 1322 may be selected to couple the primary flex interconnect 1316 to the circuit board 1320.

Furthermore, as depicted in FIG. 13, the electro-acoustic modules 1302, 1304, 1306 may be enclosed by a smooth curving material 1324, such as RTV silicone, or other material with sound speed close to 1540 m/sec. In one example, the smooth curving material may act as a lens that is disposed on the electro-acoustic modules 1302, 1304, 1306. The lens may act as a smooth surface on the electro-acoustic modules 1302, 1304, 1306, which further aids in placing the ultrasound probe on objects such as chest or abdomen of a patient.

The various embodiments of the system and method aid in forming the transducer arrays that are interchangeable and adaptable to a shape of an ultrasound probe. Moreover, these transducer arrays can be assembled on a convex structure without bending electronics or ASIC, which in turn improves the functionality and/or reliability of the ASIC. In addition, these transducer arrays are formed with minimal scrap material, and thus reducing the cost of the ultrasound probe.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An ultrasound transducer array for an ultrasound probe, comprising:
a support structure; and
a plurality of electro-acoustic modules coupled to the support structure, wherein each of the plurality of electro-acoustic modules comprises at least one matrix acoustic array and an interconnect element,
wherein each of the plurality of electro-acoustic modules is interchangeable on the support structure so as to adapt to one or more shapes of the ultrasound probe, and
wherein each of the plurality of electro-acoustic modules operates in a manner substantially identical to each other of the plurality of electro-acoustic modules.

2. The ultrasound transducer array of claim 1, wherein at least a portion of the support structure is planar.

3. The ultrasound transducer array of claim 1, wherein at least a portion of the support structure is non-planar.

4. The ultrasound transducer array of claim 3, wherein the at least one matrix acoustic array and the interconnect element are configured to conform to a shape of the non-planar support structure.

5. The ultrasound transducer array of claim 3, wherein the interconnect element having variable thickness is coupled to the at least one matrix acoustic array so as to conform each electro-acoustic module to the shape of the non-planar support structure.

6. The ultrasound transducer array of claim 1, wherein each of the plurality of electro-acoustic modules is detachably coupled to the support structure.

7. The ultrasound transducer array of claim 1, wherein each of the plurality of electro-acoustic modules is interchangeably coupled to the support structure.

8. The ultrasound transducer array of claim 1, wherein each of the plurality of electro-acoustic modules is aligned on the support structure to conform to a predetermined shape of the ultrasound probe.

9. The ultrasound transducer array of claim 1, wherein each of the electro-acoustic modules comprises a bottom surface adjacent to the support structure and a top surface opposite the bottom surface and positioned away from the support structure, and at least two beveled sides each forming a surface extending between the bottom surface and the top surface, wherein a first width of an electro-acoustic module measured near the bottom surface is less than a second width of the electro-acoustic module measured near the top surface.

10. The ultrasound transducer array of claim 1, wherein each of the plurality of electro-acoustic modules further comprises an integrated acoustic backing coupled to a heat sink, wherein the heat sink and the integrated acoustic backing are configured to absorb heat generated in the electro-acoustic modules.

11. The ultrasound transducer array of claim 1, wherein the matrix acoustic array comprises a plurality of stack elements at least partially separated by a vertical gap, wherein at least one narrow stack element is positioned between two wide stack elements, wherein the at least one narrow stack element has a width extending horizontally between the vertical gaps that is lesser than a width of the wide stack elements.

12. The ultrasound transducer array of claim 11, wherein the two wide stack elements are disposed on two sides of the matrix acoustic array such that the two wide stack elements overhang an ASIC in a corresponding electro-acoustic module.

13. The ultrasound transducer array of claim 1, wherein the matrix acoustic array comprises:
a plurality of first pads coupled between a plurality of stack elements and the interconnect element; and
a plurality of second pads coupled between an ASIC bump and the interconnect element, wherein a pitch of at least one of the first pads, the second pads, the ASIC bump, and a dicing cut is varied by a predefined amount so that the matrix acoustic array overhang the ASIC.

14. The ultrasound transducer array of claim 13, wherein the stack elements have uniform size irrespective of the pitch of the at least one of the first pads, the second pads, the ASIC bump, and the dicing cut.

15. The ultrasound transducer array of claim 1, wherein the plurality of electro-acoustic modules are enclosed by a smooth curving material.

16. The ultrasound transducer array of claim 1, wherein at least one of the electro-acoustic modules remains coupled to at least one other of the electro-acoustic modules.

17. An electro-acoustic module for an ultrasound transducer array, comprising:
a base unit comprising an acoustic backing and a heat sink, wherein the heat sink is configured to detachably couple to a support structure of the ultrasound transducer array;
an ASIC layer individually coupled to the base unit;
a flex interconnect disposed on the ASIC layer and electrically coupled to a circuit board; and
a matrix acoustic array disposed on the flex interconnect and comprising a plurality of stack elements at least partially separated by a vertical gap, wherein at least one narrow stack element is positioned between two wide stack elements, wherein the at least one narrow stack element has a width extending horizontally between the vertical gaps that is lesser than a width of the wide stack elements.

18. The electro-acoustic module of claim 17, wherein the matrix acoustic array comprises:
a plurality of first pads coupled between the stack elements and the flex interconnect; and
a plurality of second pads coupled between an ASIC and the flex interconnect, wherein a pitch of the first pads is larger than a pitch of the second pads so as to obtain the wide stack elements when the acoustic array is diced.

19. The electro-acoustic module of claim 17, wherein the heat sink comprises at least one threaded aperture for receiving at least one protruding member from the support structure.

20. The electro-acoustic module of claim 17, wherein the heat sink comprises at least one aperture for receiving at least one pin from the support structure.

21. The electro-acoustic module of claim 20, wherein the at least one pin allows the electro-acoustic module to align to the support structure.

22. The electro-acoustic module of claim 17, wherein the flex interconnect comprises:
a primary flex interconnect disposed on the ASIC layer; and
a secondary flex interconnect electrically coupled to the primary flex and the circuit board.

23. The electro-acoustic module of claim 22, wherein the secondary flex interconnect is configured to couple the primary flex interconnect to the circuit board independent of a position of the electro-acoustic module on the ultrasound transducer array.

24. The electro-acoustic module of claim 17, wherein the two wide stack elements are disposed on two sides of the matrix acoustic array such that the two wide stack elements overhang the ASIC layer.

25. The electro-acoustic module of claim 17 further comprising a lens disposed on the matrix acoustic array.
